(19) United States
(12) Patent Application Publication
(10) Pub. No.: US 2015/0168386 A1
(43) Pub. Date: Jun. 18, 2015

(54) METHOD AND SYSTEM FOR IDENTIFYING LEAKY LUNG EPITHELIAL BARRIER

(71) Applicant: Steve Nicholas Georas, Rochester, NY (US)

(72) Inventor: Steve Nicholas Georas, Rochester, NY (US)

(21) Appl. No.: 14/534,741

(22) Filed: Nov. 6, 2014

Related U.S. Application Data
(60) Provisional application No. 61/900,865, filed on Nov. 6, 2013.

(51) Int. Cl. G01N 33/53; H01J 49/00; A61K 31/047

(52) U.S. Cl. CPC G01N 33/5308; A61K 31/047; H01J 49/0027; G01N 2800/12; G01N 2800/122

(57) ABSTRACT

A method whereby a patient inhales a fixed dose of a test agent, followed by collection of a blood or urine sample, and measurement of the test agent in blood or urine. This “Leaky Lung Test” is useful in the diagnosis and management of subjects with asthma, cystic fibrosis, COPD, lung cancer and other related lung diseases. In addition, the Leaky Lung Test is useful in identifying subjects with dysfunctional epithelial barriers caused by exposure to noxious inhaled irritants, or after respiratory tract viral infections.

### BACKGROUND OF THE INVENTION

1. FIELD OF THE INVENTION

The present invention relates to a method for non-invasively measuring airway epithelial barrier function in human subjects. Airway epithelial barrier function is thought to be dysfunctional in lung diseases including asthma, cystic fibrosis, chronic obstructive pulmonary disease (COPD) and lung cancer, resulting in “leaky lungs”. By standardizing an approach to measure airway epithelial barrier function, the present invention provides a novel diagnostic and prognostic test for asthma, cystic fibrosis, COPD, lung cancer and related conditions.

2. Description of the Background

Airway epithelial cells comprise a key barrier to the outside world. The epithelial barrier includes both acellular components (e.g. mucus) as well as specialized epithelial junctional complexes between neighboring cells. These junctional complexes, consisting of tight junctions and adherens junctions, regulate the paracellular passage of molecules and ions (and also prevent lateral movements of molecules within the cellular membrane) (1, 2). When the integrity of epithelial junctions is compromised, pathology ensues. Recent research in cell culture model and animal models suggests that defective epithelial junction function is a key component of airway diseases such as asthma, cystic fibrosis, COPD, and lung cancer (3-9) (reviewed in (10, 11)).

There are currently no well-established approaches to measure the function of airway epithelial junctions in human subjects (that is, to measure “outside/in” permeability). Outside/in in this context refers to translocation of inhaled particles from the airway lumen into the sub-epithelial space, then into lymph and the blood stream, and ultimately excretion in the urine. In research settings, airway epithelial cells can be obtained from the lower airway by biopsy or brushing (5). Although useful for research purposes, this approach of obtaining airway epithelial cells is invasive (e.g. requiring bronchoscopy with sedation) and does not study epithelial cells in their tissue context. Consequently, this approach is not practical for widespread use.

The cause(s) of leaky airways in humans is/are not currently known. One possibility is that this is due to inhalation of noxious substances that disrupt the integrity of epithelial barriers, such as air pollution and cigarette smoke. Another possibility is that respiratory viruses cause dysfunction of epithelial barriers, which may be a strategy used by viruses to promote their replication.

### SUMMARY OF THE INVENTION

The invention includes a non-invasive way to measure airway epithelial barrier function in human subjects by measuring the absorption and excretion of a test agent. This invention is referred to as “The Leaky Lung Test.” To be a useful for this purpose, a test agent should have the following three properties. First, it should be safe when inhaled and not cause significant lung toxicity. Second, it should be stable and not broken down or metabolized in the airway, blood stream, or urine. Third, it should move across the airway epithelium via paracellular routes (i.e. between cells, therefore reflecting the integrity of tight junctions between cells). One test agent that fulfills each of these three criteria is mannitol. Mannitol is a simple sugar alcohol widely used as a food additive and considered “Generally Recognized as Safe.”

Inhaled mannitol is an approved agent for use in bronchoprovocation testing (14). Bronchoprovocation testing is used to assess and quantify airway hyper-reactivity, which is a hallmark of asthma (15). Bronchoprovocation testing requires a subject to inhale the provocative substance at increasing doses until in the case of a positive test the forced expiratory volume in 1 second (FEV1) decreases from baseline as determined using spirometry.

The present invention is different from bronchoprovocation because: (i) mannitol and other test substances are administered at doses below those that provoke bronchoconstriction in most subjects, (ii) mannitol or other test substances are not administered in a dose-escalating manner, (iii) the present invention specifically measures absorption and/or excretion of the test substance after inhalation.

Therefore, the present invention includes a method whereby a patient inhales a fixed dose of mannitol or other test agents fulfilling the three properties above, followed by collection of a blood or urine sample and measurement of the test agent in blood or urine. The invention will be useful in the diagnosis and management of subjects with asthma, cystic fibrosis, COPD, lung cancer and other related lung diseases. In addition, the invention will be useful in identifying subjects with dysfunctional epithelial barriers caused by exposure to noxious inhaled irritants, such as air pollution and cigarette smoke, or after respiratory tract viral infections.

### BRIEF DESCRIPTION OF THE DRAWINGS

FIG. 1 demonstrates the result of in vitro experiments using human bronchial epithelial monolayers in which the flux of mannitol across the monolayer is shown to be non-saturable.

FIG. 2 demonstrates proof-of-principle that excretion of orally inhaled mannitol is higher in a mouse model of respiratory syncytial virus (RSV) infection.

FIG. 3 is a schematic of the sequence of events in the Leak Lung Test.

FIG. 4 demonstrates results of preliminary data in which 2 healthy control subjects and 6 subjects with asthma symptoms were challenged with 635 mg inhaled mannitol, and mannitol clearance was measured in urine collected over the ensuing 24 hours using mass spectrometry.

### DETAILED DESCRIPTION OF THE INVENTION

This invention pertains to the use of inhaled test agents to measure outside/in airway permeability in human subjects using test agents that fulfill the three criteria of: (i) safety, (ii) stability, and (iii) paracellular movement.

A general outline of the Leaky Lung Test is as follows. Prior to undergoing testing, subjects will be asked to empty their bladders of all urine. In the case of test agents also present in dietary sources (e.g., mannitol), subjects will be asked to avoid consumption of those dietary sources for 6-12 hours prior to administration, and also following administration during the specimen collection period. Subjects will be administered the test agent via oral inhalation as described in more detail below. During the specimen collection period, subjects will be asked to expectorate (and not swallow) any oral secretions or airway mucus. Subsequently, blood and/or urine samples will be collected for up to 48 hours after challenge and analyzed for the test agent. Absorption and clearance of the test agent will be determined as described in more detail below.

Two general approaches can be used to administer the test agent via oral inhalation: (i) aerosolization, or (ii) dry powder inhaler. Aerosolization can be accomplished using nebulization or via metered dose inhaler. Because particle size affects deposition within the respiratory tract (16, 17), different sized particles will be used to determine permeability at different anatomic locations within the lung. Particle sizes between 5 and 10 microns will deposit in the larger airways, and particle sizes <1 micron will deposit in the smaller airways and alveoli. The size of the particle generated will be controlled by physical properties of the aerosolization device or inhaler, together with surface chemistry of the test agent.

Because breathing patterns affect the site of deposition of orally inhaled particles within the respiratory tract, different breathing patterns during administration of the test agent will be used to vary deposition of the test agent within the lung. Instructing the patent to inhale with slow deep breaths will promote penetration of inhaled particles deep into the lung targeting small airways and alveoli, whereas rapid shallow breath inhalation will target larger airways.

By measuring the concentration of the test agent in blood over time, standard pharmacokinetic/pharmacodynamic modeling will be used to infer absorption patterns across the respiratory epithelium. Peak plasma concentration will occur within a few hours, and a faster rate of rise and higher peak concentration achieved will reflect greater degrees of lung leakiness. The total amount absorbed over time (calculated by integrating concentration over time) will also be directly proportional to degree of lung leakiness.

By measuring the concentration of the test agent in urine over time, standard pharmacokinetic/pharmacodynamics modeling can be used to infer excretion patterns and clearance. Urinary clearance will be determined by multiplying the test agent concentration by total amount of urine collected over 24-48 hours. Normalization to urinary creatinine may be useful to account for water excretion during the time of measurement. Greater urinary clearance will be indicative of greater degrees of airway leakiness.

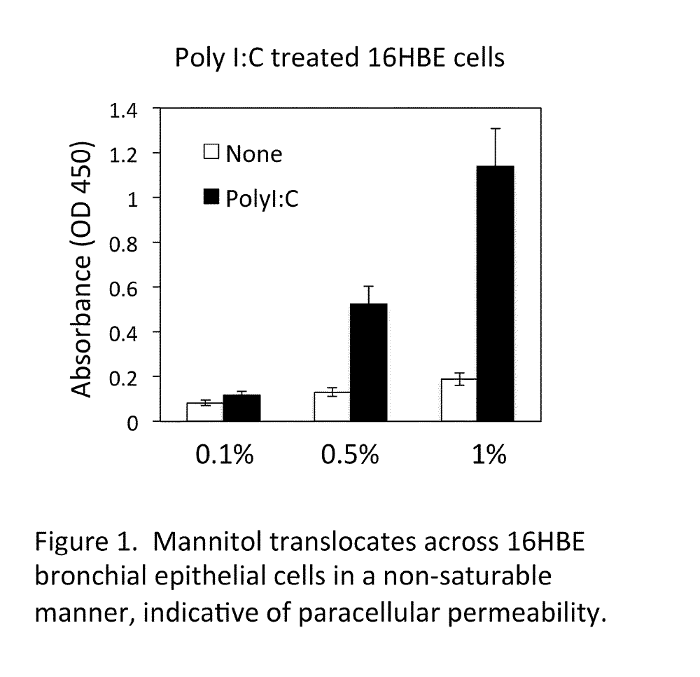

FIG. 1 demonstrates the result of in vitro experiments using human bronchial epithelial monolayers in which the flux of mannitol across the monolayer is shown to be non-saturable. The x-axis indicates the concentration of mannitol applied to the upper chamber of 16HBE cells grown to confluence on a semipermeable membrane. The y-axis indicates the concentration of mannitol detected in the lower chamber after 1 hour, determined using a colorimetric assay (where OD refers to optical density). 16HBE cells were either unstimulated (open bars) or pretreated with the viral mimetic PolyI:C (closed bars) for 18 hours prior to analysis of mannitol permeability. Note that in both unstimulated and PolyI:C-stimulated cells, the mannitol concentration detected in the lower chamber increased steadily with the concentration of mannitol applied to the top chamber (i.e. there was no plateau). This is indicative of movement across the monolayer via paracellular routes (as opposed to receptor mediated transport, for example). This demonstrates that mannitol crosses the epithelial membrane in the spaces between cells, rather than through cells.

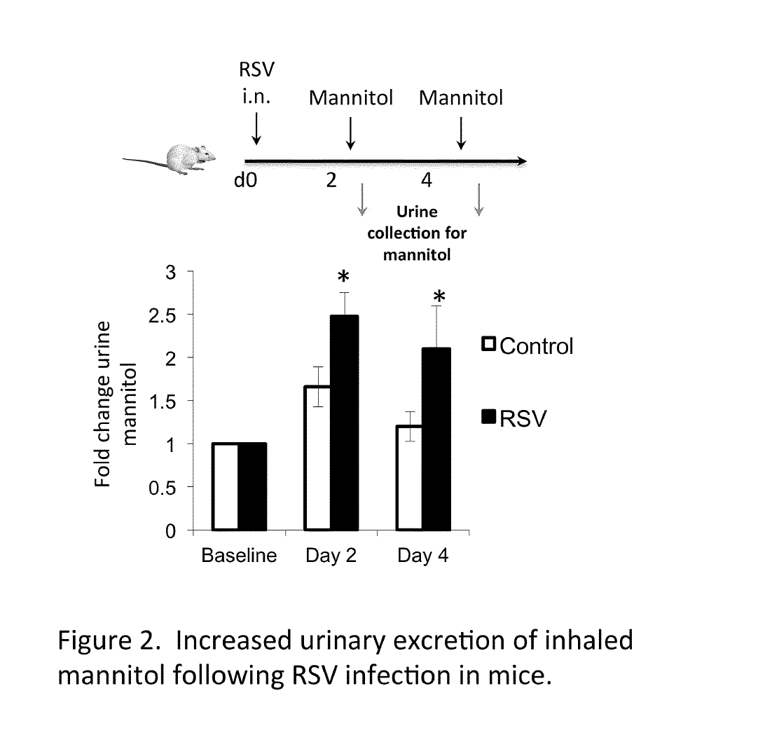

FIG. 2 demonstrates proof-of-principle that excretion of orally inhaled mannitol is higher in mice infected with respiratory syncytial virus (RSV) infection, as compared to control uninfected animals. In this experiment, 6-8 week old C57BL/6 mice were infected with a clinical isolate of RSV 9 (strain A2) intranasally (i.n.) on day 0 (d0), and then challenged with orally inhaled mannitol on days 2 and 4 as indicated by the arrows in the schematic (top of figure). Two hours after each mannitol inhalation, urine was collected from mice by scruffing, and analyzed for mannitol using a colorimetric assay. They are expressed as fold change in urine mannitol relative to uninfected control mice on d0, which was set at 1. Data are mean±standard error of the mean, of n=5-6 mice per group. * indicated p<0.05. This is the first direct demonstration that transit across the respiratory epithelium is increased following RSV infection, indicative of a leaky airway.

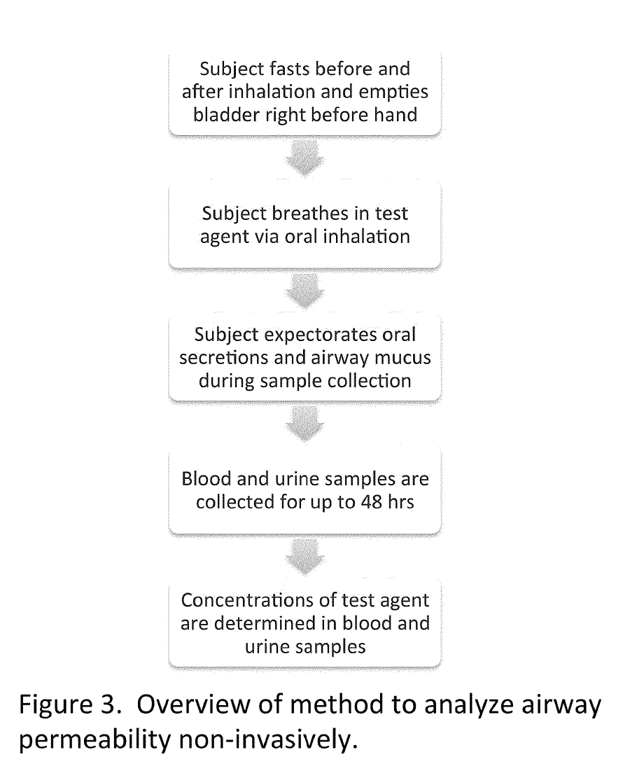

FIG. 3 is a schematic of the sequence of events in the Leak Lung Test.

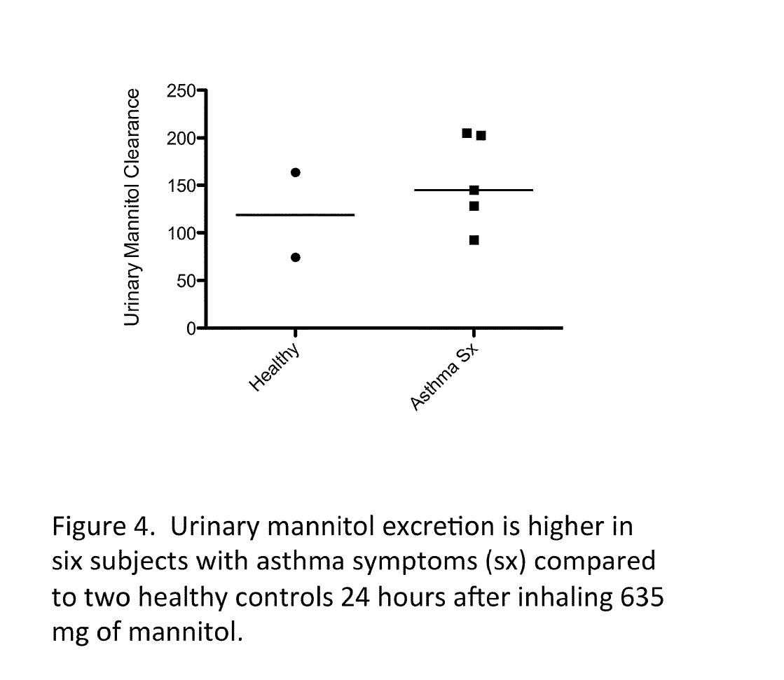

FIG. 4 demonstrates results of a study in which 2 healthy control subjects and 6 subjects with asthma symptoms were challenged with 635 mg inhaled mannitol, and mannitol clearance was measured in urine collected over the ensuing 24 hours using mass spectrometry. Results are expressed as urinary mannitol clearance, or the concentration in the urine samples collected multiplied by the amount of urine produced. Subjects with asthma symptoms had higher mannitol clearance on average. The horizontal bar indicates mean values.

The following representative examples show how the Leaky Lung Test can be used in clinical practice.

### Example 1

A Patient with Asthma Symptoms but Negative Bronchoprovocation Testing

Patient 1 is asked to empty their bladder and fast for 6 hours, and avoid foods containing mannitol for 24 hours after testing. They are then administered one inhalation of mannitol using a nebulizer device generating particle sizes averaging 5 microns, using a rapid shallow breathing pattern. This method will maximize deposition in the larger airways, the site of disease pathology in asthma. Urine will be collected for the ensuing 24 hours, and mannitol clearance calculated. Higher urinary clearance of mannitol compared to healthy control subjects will demonstrate the presence of leaky airways, suggestive of the diagnosis of asthma.

### Example 2

### A Patient with 30 Pack-year Smoking History Concerned about COPD and Lung Cancer

Patient 2 is asked to empty their bladder and fast for 6 hours, and avoid foods containing mannitol for 24 hours after testing. They are then administered one inhalation of mannitol using a dry powder inhaler generating particle sizes <1 microns with a slow deep breath. This method will maximize deposition in the smaller airways, the site of disease pathology in COPD and lung cancer. Urine will be collected for the ensuing 24 hours, and mannitol clearance calculated. Higher urinary clearance of mannitol compared to healthy control subjects will demonstrate the presence of decreased barrier function in small airway epithelial cells. Since decreased barrier function in airway epithelial cells may be a risk factor for COPD and lung cancer, this patient will be intensively counseled about smoking cessation and referred for further testing.

### Example 3

### A Young Adult with Cystic Fibrosis and Normal Lung Function

Patient 3 is asked to fast for 6 hours, and then administered one inhalation of mannitol using a dry powder inhaler generating particle sizes averaging 5 microns. Blood samples will be obtained 0.5, 1, 2 and 4 hours after testing and mannitol concentrations in plasma will be measured. Compared to other patients with cystic fibrosis, this patient demonstrates much more rapid rise and higher peak plasma concentration of mannitol, indicative of leaky airways. This patient is counseled about strategies to control airway inflammation and infection to promote healing of the dysfunctional airways.

Patient 4: a child recovering from RSV bronchiolitis

Patient 4 is asked to empty their bladder before hand, and avoid foods containing mannitol for the duration of testing. The patient is then asked to inhale a test agent using a nebulizer device generating particle sizes of 5 microns on average. Urine is collected for 24 hours and clearance of the test agent is calculated and compared to healthy age-matched control subjects. Significantly higher urinary clearance of the test agent will indicate ongoing airway leakiness, and a greater risk for subsequent lung inflammation caused by inhaled environmental pollutants and allergens. The child and her parents will be counseled to avoid exposure to environmental pollutants and allergens, until lung leakiness returns to normal.

### REFERENCES

1. Anderson, J. M., and C. M. Van Itallie. 1995. Tight junctions and the molecular basis for regulation of paracellular permeability. Am J Physiol 269: G467-475.

2. Shen, L., C. R. Weber, D. R. Raleigh, D. Yu, and J. R. Turner. 2010. Tight Junction Pore and Leak Pathways: A Dynamic Duo. Annu Rev Physiol.

3. Godfrey, R. W., N. J. Severs, and P. K. Jeffery. 1993. Structural alterations of airway epithelial tight junctions in cystic fibrosis: comparison of transplant and postmortem tissue. Am J Respir Cell Mol Biol 9: 148-156.

4. Heijink, I. H., D. S. Postma, J. A. Noordhoek, M. Broekema, and A. Kapus. 2010. House dust mite-promoted epithelial-to-mesenchymal transition in human bronchial epithelium. Am J Respir Cell Mol Biol 42: 69-79.

5. Xiao, C., S. M. Puddicombe, S. Field, J. Haywood, V. Broughton-Head, I. Puxeddu, H. M. Haitchi, E. Vernon-Wilson, D. Sammut, N. Bedke, C. Cremin, J. Sones, R. Djukanovic, P. H. Howarth, J. E. Collins, S. T. Holgate, P. Monk, and D. E. Davies. 2011. Defective epithelial barrier function in asthma. J Allergy Clin Immunol 128: 549-556 e512.

6. Hackett, T. L. 2012. Epithelial-mesenchymal transition in the pathophysiology of airway remodelling in asthma. Curr Opin Allergy Clin Immunol 12: 53-59.

7. Soini, Y. 2012. Tight junctions in lung cancer and lung metastasis: a review. International journal of clinical and experimental pathology 5: 126-136.

8. Sato, M., D. S. Shames, and Y. Hasegawa. 2012. Emerging evidence of epithelial-to-mesenchymal transition in lung carcinogenesis. Respirology 17: 1048-1059.

9. Schamberger, A. C., N. Mise, J. Jia, E. Genoyer, A. O. Yildirim, S. Meiners, and O. Eickelberg. 2014. Cigarette Smoke-Induced Disruption of Bronchial Epithelial Tight Junctions is Prevented by Transforming Growth Factor-Beta. Am J Respir Cell Mol Biol 60: 1040-1052.

10. Rezaee, F., and S. N. Georas. 2014. Breaking Barriers: New Insights Into Airway Epithelial Barrier Function in Health and Disease. Am J Respir Cell Mol Biol 50: 857-869.

11. Georas, S. N., and F. Rezaee. 2014. Epithelial barrier function: At the front line of asthma immunology and allergic airway inflammation. J Allergy Clin Immunol 134: 509-520.

12. Rezaee, F., N. Meednu, J. A. Emo, B. Saatian, T. J. Chapman, N. G. Naydenov, A. De Benedetto, L. A. Beck, A. I. Ivanov, and S. N. Georas. 2011. Polyinosinic:polycytidylic acid induces protein kinase D-dependent disassembly of apical junctions and barrier dysfunction in airway epithelial cells. J Allergy Clin Immunol 128: 1216-1224 e1211.

13. Rezaee, F., S. A. DeSando, A. I. Ivanov, T. J. Chapman, S. A. Knowlden, L. A. Beck, and S. N. Georas. 2013. Sustained protein kinase D activation mediates respiratory syncytial virus-induced airway barrier disruption. J Virol 87: 11088-11095.

14. Anderson, S. D., J. Brannan, J. Spring, N. Spalding, L. T. Rodwell, K. Chan, I. Gonda, A. Walsh, and A. R. Clark. 1997. A new method for bronchial-provocation testing in asthmatic subjects using a dry powder of mannitol. Am J Respir Crit Care Med 156: 758-765.

15. Crapo, R. O., R. Casaburi, A. L. Coates, P. L. Enright, J. L. Hankinson, C. G. Irvin, N. R. Maclntyre, R. T. McKay, J. S. Wanger, S. D. Anderson, D. W. Cockcroft, J. E. Fish, and P. J. Sterk. 2000. Guidelines for methacholine and exercise challenge testing-1999. This official statement of the American Thoracic Society was adopted by the ATS Board of Directors, July 1999. Am J Respir Crit Care Med 161: 309-329.

16. Oberdorster, G., E. Oberdorster, and J. Oberdorster. 2005. Nanotoxicology: an emerging discipline evolving from studies of ultrafine particles. Environ Health Perspect 113: 823-839.

17. Oberdorster, G., E. Oberdorster, and J. Oberdorster. 2007. Concepts of nanoparticle dose metric and response metric. Environ Health Perspect 115: A290.

1. A method for non-invasively measuring lung permeability in human subjects comprising:
administering to a subject a test agent via oral inhalation and measuring the amount of test agent in the subject's bloodstream or in the urine, wherein the test subject is non-toxic, inert, and translocates across surface epithelial cells via paracellular permeability.

2. The method according to claim 1 wherein the oral inhalation is accomplished by one of the following:
a. aerosol delivery via nebulization
b. oral inhalation via dry powder inhaler
c. oral inhalation via metered dose inhaler.

3. The method according to claim 2 further comprising determining permeability in different parts of the lung, comprising administering a test agent having particles of different size.

4. The method according to claim 2 further comprising determining permeability in different parts of the lung comprising instructing the patient to inhale with different breathing patterns to alter deposition within the respiratory tract (e.g. slow deep breathing vs. rapid shallow breathing).

5. The method according to claim 1 comprising detecting the presence of the test agent in peripheral blood plasma or serum samples.

6. The method according to claim 1 comprising detecting the presence of the test agent in collected urine samples.

7. The method according to claim 1, wherein the test agent is mannitol, and wherein the method of detecting the presence of mannitol is selected from one of the following:
a. Colorimetric assays of mannitol conversion to fructose,
b. Enzyme-linked immunosorbent assays of mannitol concentration relative to an internal standard,
c. Quantitative mass spectrometry of mannitol with an internal standard.

8. The use of claim 1 as a diagnostic test for asthma, cystic fibrosis, COPD lung cancer, and other respiratory tract diseases characterized by abnormal epithelial cells.

9. The use of claim 1 as a prognostic test for asthma, cystic fibrosis, COPD lung cancer, and other respiratory tract diseases characterized by abnormal epithelial cells.

10. The use of claim 1 as a diagnostic test of leaky airways caused by respiratory viral tract infection.

11. The use of claim 1 as a diagnostic test of leaky airways caused by cigarette smoke inhalation.

12. The use of claim 1 as a diagnostic test of leaky airways caused by allergen inhalation.
